(19) United States
(12) Patent Application Publication
(10) Pub. No.: US 2024/0148549 A1
(43) Pub. Date: May 9, 2024

(54) PENCIL GRIP VITREOUS ASPIRATION AND DRUG DELIVERY DEVICE

(71) Applicant: TWENTY TWENTY THERAPEUTICS LLC, South San Francisco, CA (US)

(72) Inventors: Alexandre R. Tumlinson, South San Francisco, CA (US); VanDee Moua, South San Francisco, CA (US); Dimitri Azar, Chicago, IL (US)

(21) Appl. No.: 18/382,411

(22) Filed: Oct. 20, 2023

Related U.S. Application Data
(60) Provisional application No. 63/417,798, filed on Oct. 20, 2022.

(51) Int. Cl. A61F 9/00

(52) U.S. Cl. CPC A61F 9/0017

(57) ABSTRACT

A vitrectomy device includes a body extending along an axis from a proximal end to a distal end and having a central passage. A needle is provided for insertion into the vitreous humor of an eye and extends within the central passage and out of the distal end of the body. An assembly including a chamber under vacuum and a piercing member extend away from the chamber. The chamber is movable in a direction transverse to the centerline from a first position in which the chamber is not fluidly connected to the needle to a second position in which the piercing member fluidly connects the chamber to the needle for drawing a vitreous sample into the chamber under vacuum pressure.

### RELATED APPLICATIONS

This application claims the benefit of U.S. Provisional Appln. Ser. No. 63/417,798, filed Oct. 20, 2022, the entirety of which is incorporated herein by reference.

### TECHNICAL FIELD

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites and nutrients. It is produced largely by cells of the ciliary body. Changes in vitreous structure that occur with aging, are important in the pathogenesis of many vitreoretinal diseases.

Intraocular pressure (IOP) quantifies the pressure of the vitreous humor inside the eye. Many individuals suffer from disorders, such as glaucoma, that are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection. Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one week. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to approximately the full depth of the needle, injecting the drug, withdrawing the needle, and allowing the conjunctiva to cover the injection site. Post injection care typically includes a basic verification of functional vision such as requesting the patient to count the number of fingers shown by the doctor. This functional test verifies that acute IOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauge may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase IOP by as much as 25 mmHg, which is substantially greater than threshold levels that are considered potentially harmful. Evidence shows that while such IOP increases are transient, they are in fact associated with an iatrogenic glaucoma resulting in measurable loss of nerve fiber layer and visual function over a course of only several treatments in patients with ‘normal’ resting IOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required.

### SUMMARY

In one example, a vitrectomy device includes a body extending along an axis from a proximal end to a distal end and having a central passage. A needle is provided for insertion into the vitreous humor of an eye and extends within the central passage and out of the distal end of the body. An assembly including a chamber under vacuum and a piercing member extend away from the chamber. The chamber is movable in a direction transverse to the centerline from a first position in which the chamber is not fluidly connected to the needle to a second position in which the piercing member fluidly connects the chamber to the needle for drawing a vitreous sample into the chamber under vacuum pressure.

In another example, a method includes inserting a sharpened distal end of a needle of a device into a globe of an eye, wherein the device includes a body extending along an axis from a proximal end to a distal end and having a central passage. A needle is provided for insertion into the vitreous humor of an eye and extends within the central passage and out of the distal end of the body. An assembly includes a chamber under vacuum and a piercing member extending away from the chamber. The chamber is moved in a direction transverse to the centerline from a first position in which the chamber is not fluidly connected to the needle to a second position in which the piercing member fluidly connects the chamber to the needle for drawing a vitreous sample into the chamber under vacuum pressure.

Other objects and advantages and a fuller understanding of the invention will be had from the following detailed description and the accompanying drawings.

### BRIEF DESCRIPTION OF THE DRAWINGS

FIG. 1 is a schematic illustration of an example combined biological sampling and injection assembly in accordance with the present invention.

FIG. 2 is an exploded view of the assembly of FIG. 1.

FIG. 3 is a section view of the assembly of FIG. 1 taken along line 3-3.

FIG. 4 is a section view of a body of the assembly of FIG. 1.

FIG. 5A is an exploded view of a portion of a vacuum sample assembly.

FIG. 5B is a section view of the portion of the vacuum sample assembly of FIG. 5A.

FIG. 6 is a section view of another portion of the vacuum sample assembly.

FIG. 7A is a front view of a syringe of the assembly of FIG. 1.

FIG. 7B is a section view of the syringe of FIG. 7A taken along line 7B-7B.

FIG. 8 in an enlarged view of a portion of the assembly prior to operation.

FIG. 9A is a schematic illustration of the assembly positioned with a package.

FIG. 9B is a schematic illustration of the assembly removed from the package.

FIG. 9C is a more detailed view of the package.

FIG. 10A is schematic illustrations of the assembly during a first step of operation.

FIG. 10B is schematic illustrations of the assembly during a second step of operation.

FIG. 10C is schematic illustrations of the assembly during a third step of operation.

FIG. 11 is a schematic illustration of a sample tube for collecting a sample from the assembly.

FIG. 12A are schematic illustrations of another example combined biological sampling and injection assembly.

FIG. 12B is schematic illustrations of the assembly of FIG. 12A during a first step of operation.

FIG. 12C is schematic illustrations of the assembly of FIG. 12A during a second step of operation.

FIG. 13A is a schematic illustration of a modified version of the example combined biological sampling and injection assembly.

FIG. 13B is schematic illustrations of the assembly of FIG. 13A during a first step of operation.

FIG. 13C is schematic illustrations of the assembly of FIG. 13A during a second step of operation.

FIG. 13D is schematic illustrations of the assembly of FIG. 13A during a third step of operation.

FIG. 13E is schematic illustrations of the assembly of FIG. 13A during a fourth step of operation.

FIG. 14 is a schematic illustration of another example combined biological sampling and injection assembly.

FIG. 15 is a schematic illustration reflecting one implementation of the assembly of FIG. 14

FIG. 16 is a schematic illustration reflecting an alternative implementation of the assembly of FIG. 14.

### DETAILED DESCRIPTION

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

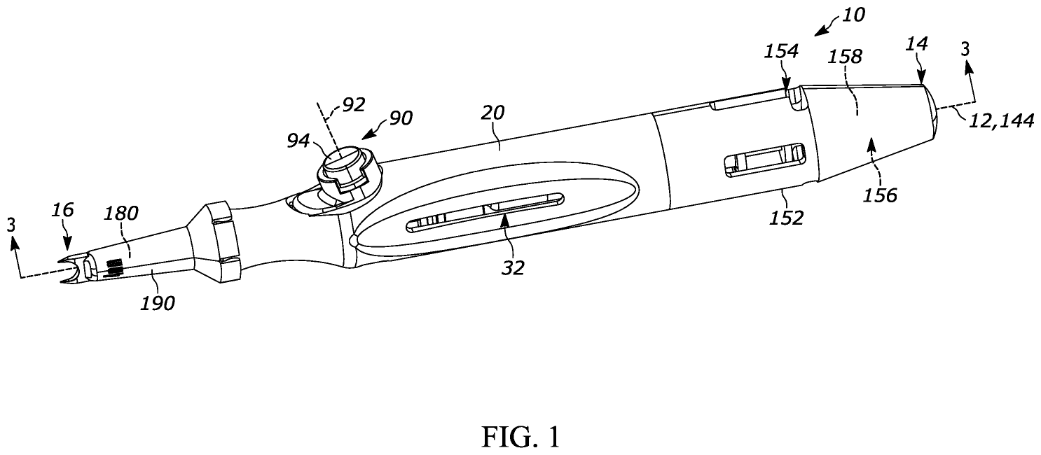

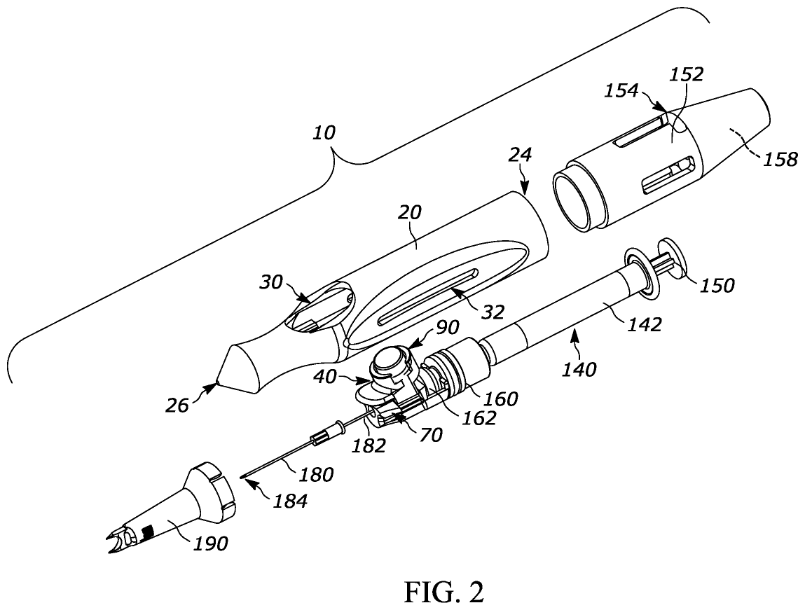

FIGS. 1-3 illustrate an example combined biological sampling and injection assembly or device 10 in accordance with the present invention. The device 10 is of a pencil-grip design and extends along a centerline 12 from a first or proximal end 14 to a second or distal end 16. The device 10 is operable by a user, e.g., a physician, to obtain a sample of vitreous humor from an eye of a patient.

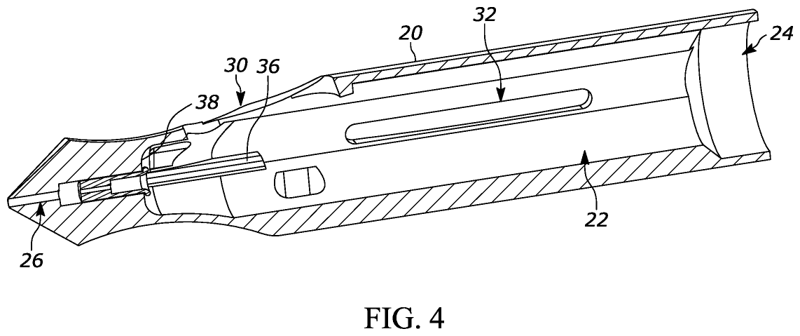

Referring to FIG. 4, the device 10 includes a tubular gripping body 20, e.g., a “pencil-grip” type body, having a central passage 22 extending generally along the centerline 12 of the device 10. A countersink 24 extends from the proximal end of the gripping body 20 to the central passage 22. A lumen 26 extends from the distal end of the gripping body 20 to the central passage 22. A lateral passage 30 extends from the periphery of the gripping body 20 to the central passage 22. One or more lateral openings 32 also extend from the periphery of the gripping body 20 to the central passage 22 and are located proximal to the lateral passage 30. A pair of guide ribs 36 (only one is shown) extends longitudinally along the interior or the gripping body 20 towards a distally located inner surface 38. The guide ribs 36 extend parallel to one another and are symmetrically arranged about the interior of the gripping body 20.

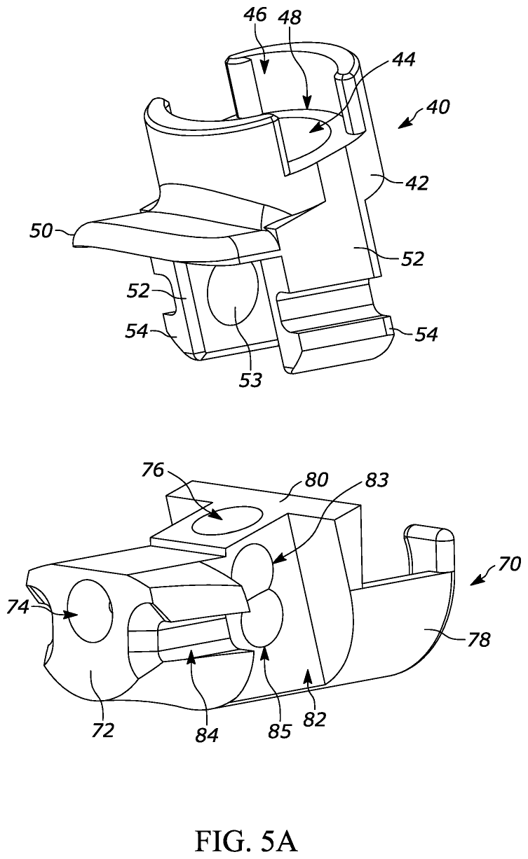

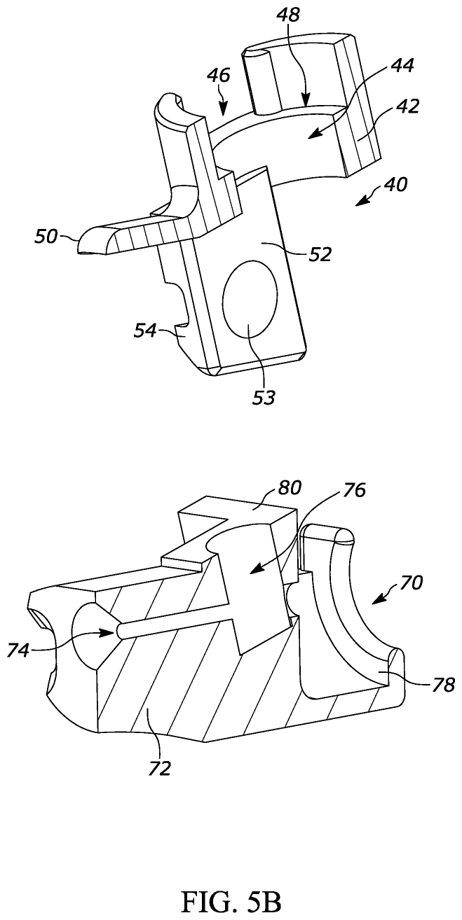

Referring to FIGS. 5A-5B, the device 10 further includes a follower 40 and advancement clip 70 that cooperate with one another to operate the device. The follower 40 includes a generally cylindrical base 42. A central passage 44 through into the base 42. A pocket 46 having a larger cross-section than the cross-section of the central passage 44 is aligned with the central passage and extends thereto. An annular recess 48 is provided within the pocket 46. A projection 50 extends distally from the base 42. A pair of legs 52 extends downward (as shown) from the base 42. Each leg 52 includes a tab or projection 54 extending outward and away from the other tab. A projection 53 is provided on each leg 52 such that the projections face/extend towards one another. In one example, the projections are hemispherical, but other shapes are contemplated.

The advancement clip 70 includes a base 72. A central lumen 74 extends through the base 72 to a laterally extending pocket 76. The pocket 76 extends from an exterior surface 80 of the base 72. A receiving member 78 extends proximally from the base 72. In one example, the receiving member 78 is an L-shaped projection.

First lateral recesses 82 extend downwardly (as shown) along either side of the base 72 on opposite sides of the pocket 76. First and second pockets 83, 85 are provided along each lateral recesses 82. The pockets 83, 85 are aligned with one another below (as shown) the surface 80. The pockets 83, 85 have substantially the same size and shape as the projections 53 on the legs 52.

Second lateral recesses 84 extend longitudinally along either side of the base 72 from the distal end to the first lateral recesses 82. The second lateral recesses 84 are configured to receive the guide ribs 36 on the gripping body 20.

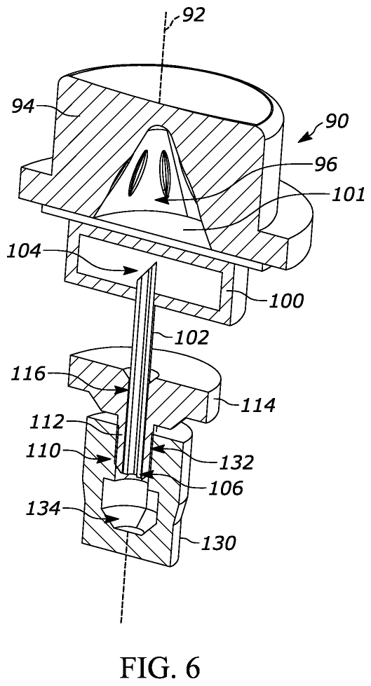

Referring to FIG. 6, a vacuum sample assembly 90 is provided for connection to the follower 40. The vacuum sample assembly 90 extends generally along a centerline 92 and includes a clear/transparent container 94 defining a sample chamber 96. The sample chamber 96 is evacuated of air and is therefore at vacuum pressure. A compliant seal 100 includes a cover or septum 101 for sealing the sample chamber 96.

The compliant seal 100 provides a short term, airtight seal against the needle 180 during the procedure, whereas the septum 101 provides long term maintenance of the vacuum in the sample chamber 96 during storage. The sample chamber 96 has a conical shape which is useful for sample handling in the laboratory, such as for centrifugation. The wall of the sample chamber 96 also has surface indents or roughnesses that cause a lensing or scattering effect when the chamber is empty. This lensing or scattering effect, however, effectively disappears when the chamber is filled with a clear fluid with an index of refraction more closely matched to the clear material of the vacuum chamber.

The vacuum sample assembly 90 has an annular lip to help removable hold the assembly in the follower 40. The annular lip also provides a reference surface to manage the assembly 90 in the lab, such as to locate it in place within a multi-well plate for centrifugation or automated sampling. The septum 101 may have an extended tab (not shown), not sealed to the container 94, which may be used in the laboratory for the purpose of easily peeling the entire seal away from the container.

A piercing member 102 extends from a first end 104 positioned within the seal 100 to a second end 106 extending into a holder 110. The holder 110 includes a cylindrical base or body 112 and a flange 114 extending radially therefrom. A central passage 116 extends through the flange 114 and the body 112 and receives the piercing member 102. The base 112 of the holder 110 is received by a compliant sealing member 130. To this end, the compliant sealing member 130 includes a longitudinally extending first passage 132 that terminates at a chamber 134. The base 112 is received by the first passage 132. A second passage 136 (not shown here) extends laterally through the compliant sealing member 130 to the chamber 134.

The compliant sealing member 130 can be manufactured by insertion into the independently molded advancement clip 70. Alternatively, the compliant sealing member 130 can be molded in place within the advancement clip 70 in an overmolding process. The piercing member 102 is adhesively bonded to the flange 114 in an airtight manner. The flange 114 is threaded to the advancement clip 70.

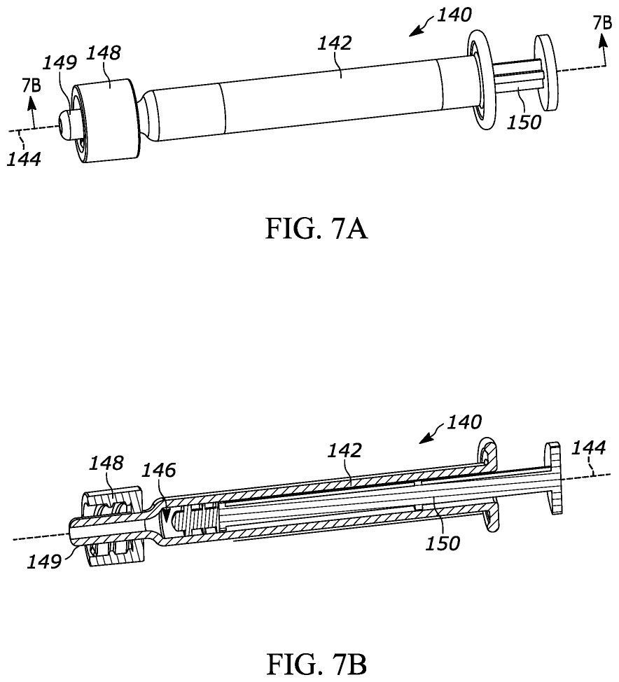

Turning to FIGS. 7A-7B, a syringe 140 is provided and includes an elongated, cylindrical body 142 extending along a centerline 144. A central passage 146 extends the length of the body 142. A coupling member 148 having, for example, a luer lock, is provided at the distal end of the body 142. A tip 149 of the guide body 142 extends through the center of the coupling member 148. A plunger 150 is provided within the central passage 146 and is longitudinally movable relative to the body 140 along the centerline 144. The plunger 150 is fluidly sealed with the inner surface of the body 140 defining the central passage 146.

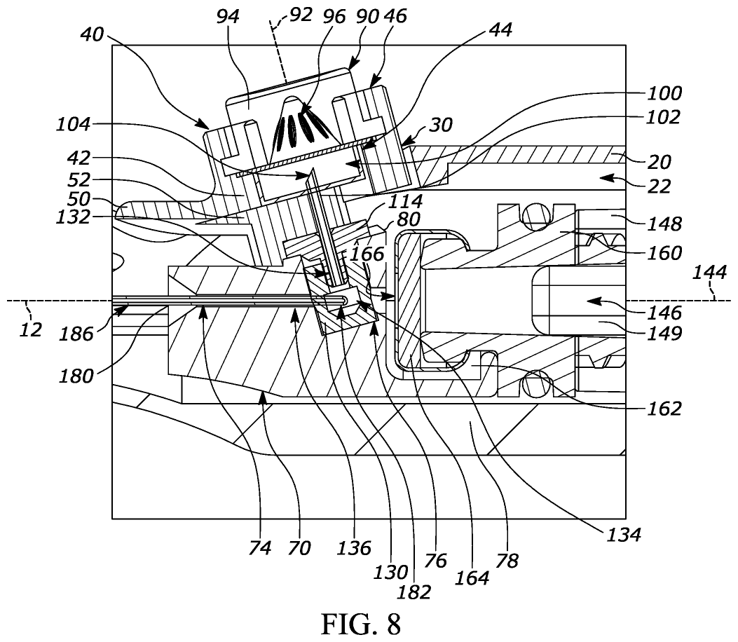

When the device 10 is assembled (see FIG. 8), the advancement clip 70 is positioned within the central passage 22 of the gripping body 20 with the lumen 76 aligned generally with the centerline 12. The guide ribs 36 of the guide body 30 extend into the second lateral recesses 84 of the advancement clip 70 to allow for sliding longitudinal movement of the advancement clip relative to the guide body.

The follower 40 is coupled to the advancement clip 70 with the legs 50 being received in the first lateral recesses 82. To this end, the follower 40 is positioned over the advancement clip 70 such that the projections 53 engage the first pockets 83 in the advancement clip 70. The coupling between the projections 53 and pockets 83 holds the follower 40 in position relative to the advancement clip 70 until a predetermined force is applied to the follower, as will be discussed. In this position, the tabs 54 on the legs 52 are aligned with the respective second lateral recesses 84 in the advancement clip 70.

The vacuum sample assembly 90 extends into the follower 40 and the advancement clip 70. In particular, the compliant sealing member 130 is positioned within the lateral pocket 76 in the advancement clip 70 with the second passage 136 of the compliant sealing member 130 aligned with the central passage 74 of the advancement clip. The flange 114 abuts the surface 80 of the advancement clip 70 and is received between the legs 52 of the follower 40.

The seal 100 is positioned within the central passage 44 of the follower 40 abutting the base 42. The container 94 is received in the pocket 44 of the follower 40. With this in mind, the centerline 92 of the vacuum sample assembly 90 extends transverse to the centerline 12 of the device 10. In other words, the container 94 and sample chamber 96 are offset from the centerline 12. The piercing member 102 extends into the seal 100 but is initially spaced from the cover 101 and sample chamber 96.

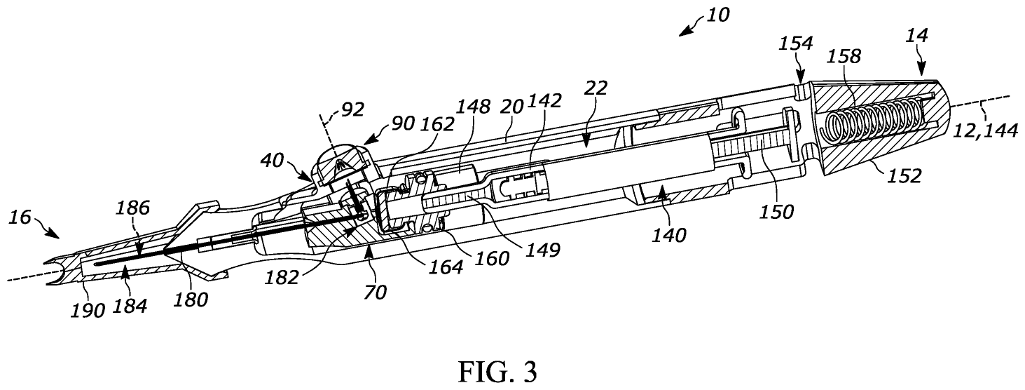

The syringe 140 is positioned within the central passage 22 of the gripping body 20 (see also FIG. 3) such that the coupling member 148 and tip 149 are positioned adjacent the advancement clip 70. A female luer-to-vial cap adaptor 160 receives the tip 149 and connects to the coupling member 148. A septum 164 is provided at the distal end of the adaptor 160 and is secured thereto by a crimped cap 162. The cap 162 includes an opening 166 exposing a portion of the septum 164 aligned with and adjacent to the central passage 74 of the advancement clip 70.

A drug capsule, such as a therapeutic agent, not shown, is provided within the adaptor 160 and tip 149 of the plunger 150 between the septum 164 and the distal end of the plunger 150. The septum 164 seals the drug capsule within this cavity, and the drug is not pressurized at this point in time. A hollow cap 152 receives the plunger 150 and includes one or more lateral passages 154. A biasing feature 158 is also received by the cap 152. In the example shown, the biasing feature is a compression spring 158. Alternatively, the biasing feature can be a gas cylinder (not shown).

A hollow eye penetration member or needle 180 extends longitudinally along the centerline 12 from a first or proximal end 182 positioned within the chamber 134 of the compliant sealing member 130 to a second or distal end 184 positioned outside the gripping body 20. The needle 180 therefore extends through the lumen 26 and central passage 22 of the gripping body 20, the central passage 74 of the advancement clip 70, and the second passage 136 of the compliant sealing member 130. The second end 182 of the needle 180 is also aligned with the opening 166 in the cap 162. A central lumen 186 extends the entire length of the needle 180. The needle 180 has a sharpened tip configured for insertion into or through at least one of the sclera, the pars plana, the anterior segment or the vitreous body of an eye.

Any gap existing between the crimped cap 162 and the advancement clip 70 can be sealed by pressing the two surfaces into contact with one another or by supplying a compliant interface (not shown) between the two to seal the space from ingress of infectious foreign material that could enter the device housing after the sterile seal is removed from the packaging.

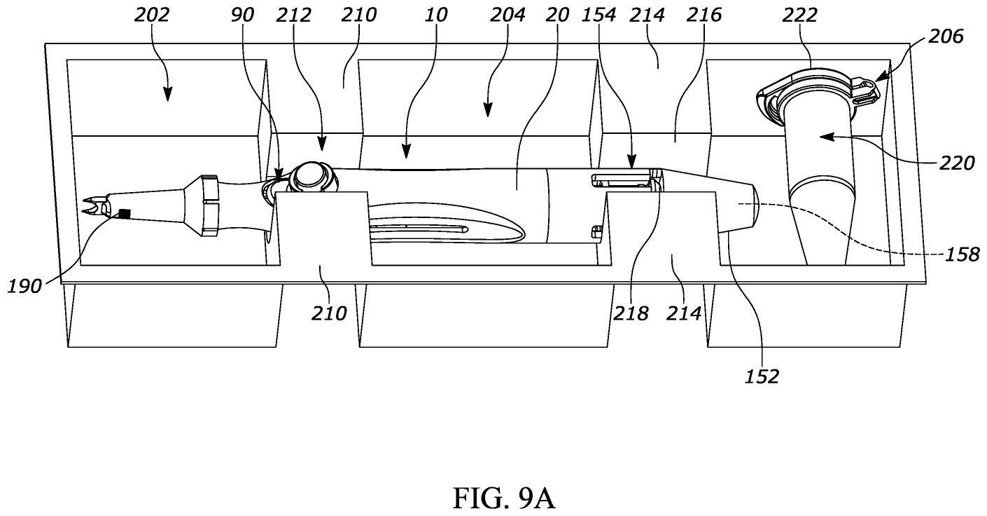

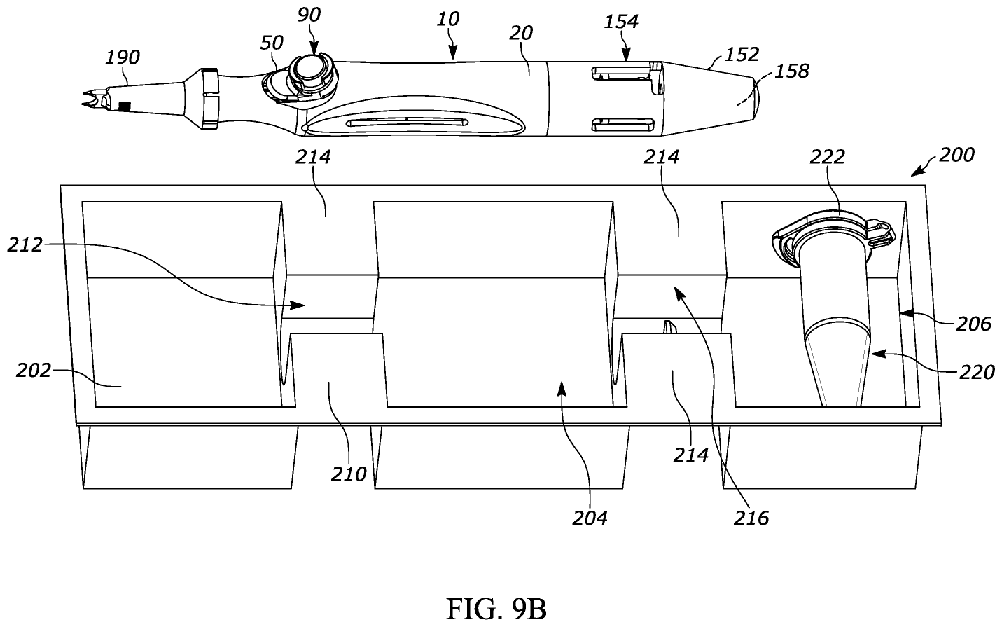

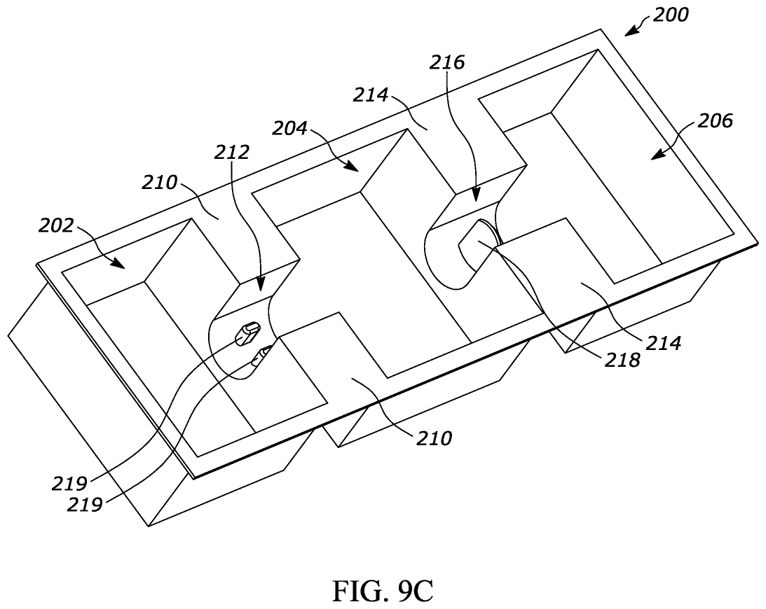

A package 200 (FIGS. 9A-9C) is provided for receiving the device 10. The package 200 includes a series of generally aligned receptacles 202, 204, 206. Projections 210 extend towards one another to demarcate the receptacles 202, 204 from one another. A passage 212 extends between the projections 210 and between the receptacles 202, 204. Projections 214 extend towards one another to demarcate the receptacles 204, 206 from one another. A passage 216 extends between the projections 214 and between the receptacles 204, 206. A projection 218 extends transverse to the projections 214 and into the passage 216. A pair of projections 219 extends transverse to the projections 210 and into the passage 212.

The package 200 is configured to help hold and transport the device 10 in a manner that prevents its unintentional activation. To this end, the device 10 is positioned within the package 200 such that the body 20 extends through the second recess 204 and adjacent passages 212, 216. With this in mind, the projections 210, 214 engage opposing sides of the body 20. The projection 218 extends into the lateral passage 154 of the cap 152 and between the end of the plunger 250 and the spring 158 (see also FIG. 3). In this manner, the projection 218 prevents the spring 158 from acting on the plunger 150.

At the same time, the projections 219 extend through corresponding openings (not shown) in the body 20 and into the lateral recesses 82 on the advancement clip 70 (see also FIG. 5A). The projections 219 thereby prevent relative sliding movement between the follower 40 and the advancement clip 70. Consequently, the projections 218, 219 on the package 200 cooperate to prevent use of the device 10 until the device is removed from the package, as will be discussed. In other words, while in the package 200 the device 10 is considered to be “disarmed” and inoperable. It will be appreciated that the package 200 can be sealed with a tyvek lid or placed within an envelope or other container to maintain a sterile environment around the device 10 until the time for use.

A sample tube 220 is provided in the third recess 206. The tube 220 includes a lid 222 and is configured to receive one of the containers 94 of the vacuum sample assembly 90 once the vitrectomy is complete. Each device 10 has a unique identifier that may be marked on the exterior of the package 200, the sample tube 220, and the vacuum sample assembly 90. This identifier may be machine readable format such as bar code, QR code, as well as human readable forms depending on the space available in each location. A mobile application or other data entry device may be used to scan the device ID on the exterior of the sterile packaging prior to the procedure to connect the device ID to the patient record. The sample tube and vacuum sample tube may be scanned at the laboratory to track the sample and connect analysis results back to the patient record.

In operation and referring further to FIG. 9B, in advance of the vitrectomy the user can remove the device 10 from the package 200. In doing so, the projection 218 is removed from the lateral passages 154 of the cap 152, which allows the spring 258 to expand and engage the piston 150. At the same time, the projections 219 are removed from the lateral passages 82 in the advancement clip 70. Consequently, the device 10 in this state is considered to be “ready” or “armed” and therefore operable.

The user can rely on the lateral openings 32 in the body 20 to ensure the drug is provided in the device 10. The user can then remove the needle cap 190 to expose the distal end 184 of the needle 180. In this configuration, the projection 50 on the follower 40 prevents dust from entering the central passage 22 via the lateral passage 30. The engagement between the ribs 36 and the legs 52 prevents the spring 158 from distally advancing the syringe 140 or vacuum sample assembly 90.

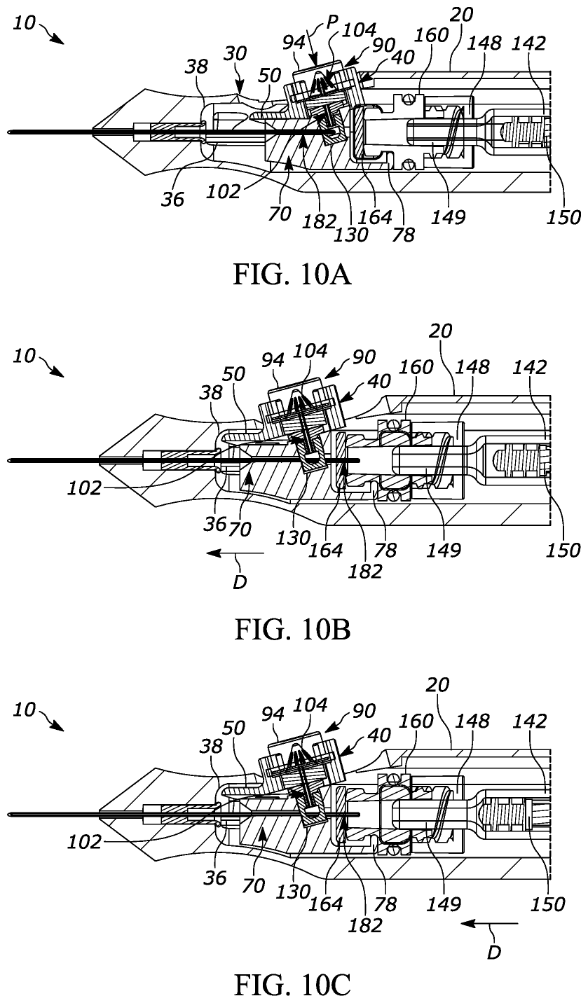

Turning to FIGS. 8 and 10A, the distal end 184 of the needle 56 is advanced into the vitreous humor of the eye (not shown). The container 94 and/or follower 40 are depressed downward and rearward (as shown) in the direction P towards the advancement clip 70. Initially, relative movement between the follower 40 and the advancement clip 70 is resisted due to the engagement between the projections 53 on the follower and the first pockets 83 on the follower (see FIG. 5A).

When sufficient force is applied in the direction P, the legs 52 on the follower 40 deflect outward and away from one another as the projections 53 disengage from the first pockets 83. The projections 53 then slide downward (as shown) along the lateral recesses 82 in the advancement clip 70 and into engagement with the second pockets 85. The engagement between the projections 53 and second pockets 85 fixes the relative positioning between the follower 40 and advancement clip 70. At the same time, the projection 50 is moved through the lateral opening 30 and into the central passage 22 of the body 20.

Moving the follower 40 downward until the projections 53 engage the second pockets 85 causes the first end 104 of the piercing member 102 to pierce the cover 101. This places the chamber 96 in fluid communication with the lumen of the piercing member. The follower 40 is held in this position by the user's thumb or index finger (not shown). This allows the vacuum within the chamber 96 to disrupt the integrity of the vitreous humor. More specifically, the vacuum breaks apart and liquefies the vitreous humor sufficient to draw portions of the vitreous humor into the lumen of the needle 186, into the chamber 134, through the piercing member 102, and ultimately into the sample chamber 96.

Once the sample chamber 96 is filled, the vacuum sample assembly 90 is distally advanced in the direction D in FIG. 10B. Advancement of the vacuum sample assembly 90 in the direction D is facilitated by the compression spring 158. In other words, once the projection 50 is moved into the central passage 22, releasing the follower 40 allows the spring 158 to automatically push the syringe 140 distally, thereby pushing the vacuum sample assembly 90 connected thereto distally in the direction D. Stated differently, as long as the user pushes the vacuum sample assembly 90 in the manner P the bias of the spring 158 is negated.

Movement of the vacuum sample assembly 90 in the direction D is guided by the cooperation of the guide ribs 36 on the body 20 with the second lateral recesses 84 in the advancement clip 70. To this end, the guide ribs 36 and recesses 84 extend parallel to the direction D. As the vacuum sample assembly 90 moves in the direction D, the first end 182 of the needle 180 passes through the chamber 134 and the compliant sealing member 130 and ultimately through the septum 164 into the adaptor 160 (FIG. 10B). This places the needle 180 in fluid communication with the drug (not shown).

The vacuum sample assembly 90 is advanced in the direction D until the projection 50 [or the advancement clip 70] abuts the inner surface 38 of the body 20. This prevents further movement of the vacuum sample assembly 90 in the direction D and, thus, prevents further movement of the adaptor 160 and syringe body 142 connected thereto in the direction D. The plunger 150, however, is capable of sliding movement in the direction D relative to the body 142 and under the bias of the spring 158.

With this in mind, once the projection 50 abuts the inner surface 38, the spring 158 causes additional advancement of the plunger 150 in the direction D relative to body 142 of the syringe 140 (FIG. 10C). That said, the advancing plunger 150 delivers the drug (not shown) provided within the adaptor 160. To this end, the advancing plunger 150 pressurized and pushes the drug through the tip 149, into the first end 182 of the needle 180 within the adaptor 160, and ultimately out of the second end 184 of the needle to be fully injected into the eye where the vitreous humor 14 sample was removed. The adaptor 160 and/or coupling member 148 can be formed from a transparent or semi-transparent material to enable the user to confirm that the drug has been delivered by looking through the lateral openings 32 in the body 20 and/or the lateral passages 154 in the cap 152.

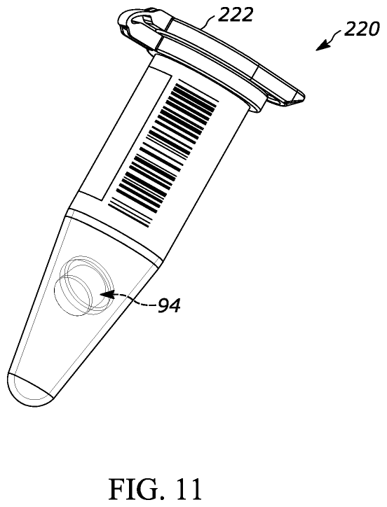

Once drug delivery is complete, and the device 10 is withdrawn from the eye, the container 94 bearing the removed vitreous humor can be removed from the follower 40 and placed within the sample tube 220 (FIG. 11). The seal 100 automatically closes around the puncture location and prevents leakage of the acquired sample. The sample tube 220 is then placed in a refrigerated space, while the remainder of the device 10 and any contaminated sharps may be discarded. With this in mind, the sample tube 220 can include a cold-sensitive label that indicates if the temperature of the sample tube exceeds predetermined conditions, e.g., about 8*C for more than about 60 minutes.

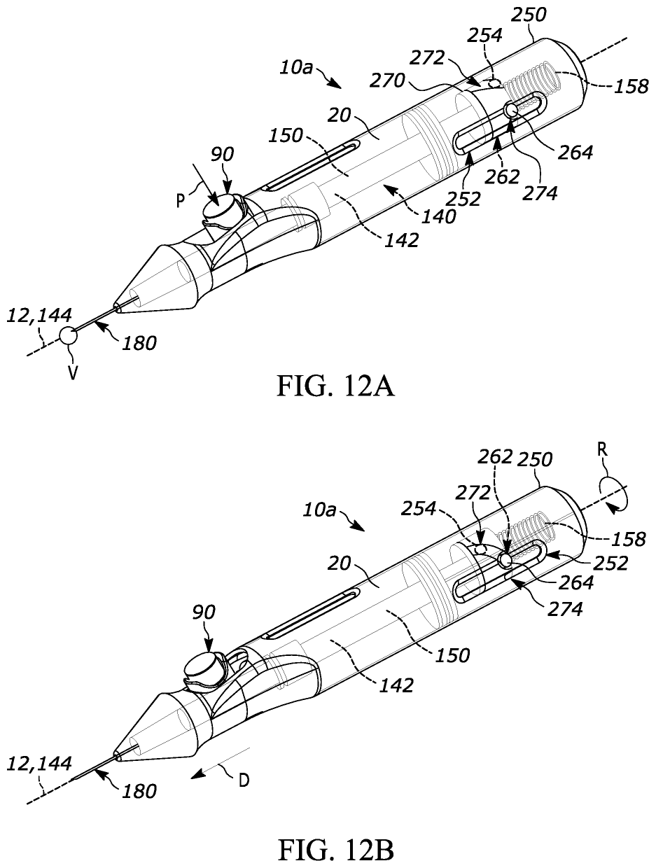

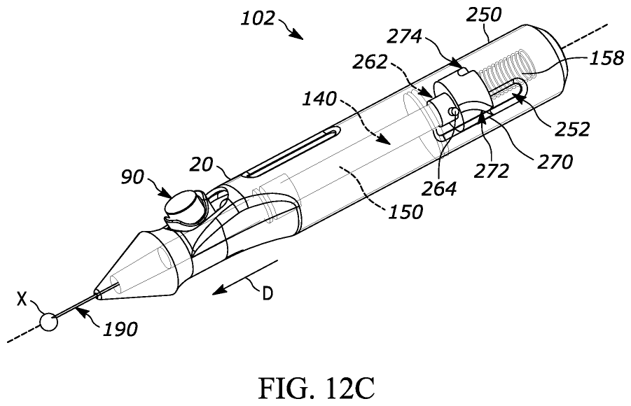

Another example device 10a is illustrated in FIGS. 12A-12C. Features in FIGS. 12A-12C that are identical to features in FIGS. 1-10C are given the same reference number. In FIGS. 12A-12C, the device 10a includes a cam system for helping to control advancing of the syringe and plunger in a manner that delays pressurizing the drug until the moment of delivery. To this end, the device 10a includes a cap 250 having longitudinally extending slots 252. At least one pin or projection (shown in phantom at 254) extends radially inward from the cap 250.

A radial collar 270 is rotatably connected to the body 142 adjacent to a proximal end 262 of the plunger 150. At least one helical groove 272 is provided in the collar 270 and configured to receive a corresponding pin 254. A slot 274 also extends into the collar 270 and is misaligned from the groove 272. The slot 274 receives a projection 264 on the end 262 of the plunger 150.

During operation, the vacuum sample assembly 90 is depressed and held in the manner P to automatically draw in the vitreous sample, indicated at V in FIG. 12A as previously described. Releasing the vacuum sample assembly 90 allows the spring 158 to bias the syringe 140 in the direction D along the centerlines 12, 144, as shown in FIG. 12B. The syringe 140, in turn, pushes the vacuum sample assembly 90 in the direction D until the projection 50 abuts the inner surface 38.

When the syringe 140 moves, the plunger 150 and body 142 initially move together without relative axial movement therebetween. To this end, the collar 270 abuts the projection 264 on the plunger 150 to initially prevent movement of the plunger into the body. At the same time, axial movement of the collar 270 [with the body 142] causes the projection 254 on the cap 250 to slide within the corresponding helical groove 272 in the collar. This, in turn, causes the collar 270 to rotate in the manner R about the centerlines 12, 144 while moving axially in the direction D.

The helical groove 274 is configured such that the pin 254 reaches the distal end of the groove just prior to the axial movement D of the syringe 140 terminating. With that said, as the projection 50 abuts the inner surface 38 to stop axial movement D of the syringe 140, the groove 274 rotates into alignment with the projection 264 on the plunger 260 (FIG. 12B). When this occurs, the proximal end 262 of the plunger 150 is now capable of moving longitudinally relative to the body 142 and rotating collar 270. In particular, the spring 158 now pushes the plunger 150 into the body 142 in the direction D as the projection 264 slides through the slot 274, which rotates with the collar 270. Consequently, the plunger 150 pushes the drug out of the needle 180 and into the eye, as indicated at X in FIG. 12C.

Configuring the device 10a in this manner is advantageous because the collar 270 prevents the plunger 150 from pressurizing the drug until the time of (or just prior to) delivery to the eye. Delaying the pressurization of the drug helps to mitigate leakage of the drug from the device 10a prior to the intended delivery time on account of, for example, handling of the device or machine tolerances in the assembled parts of the device.

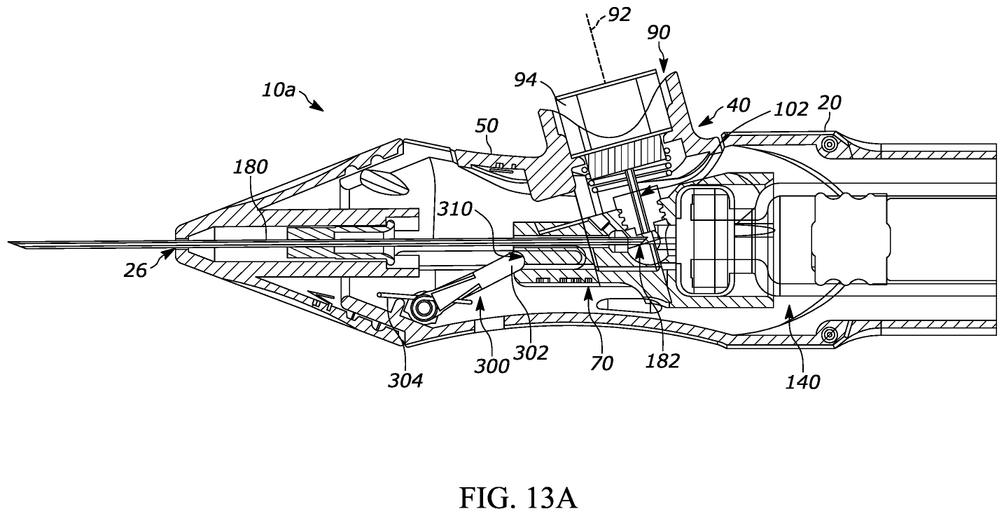

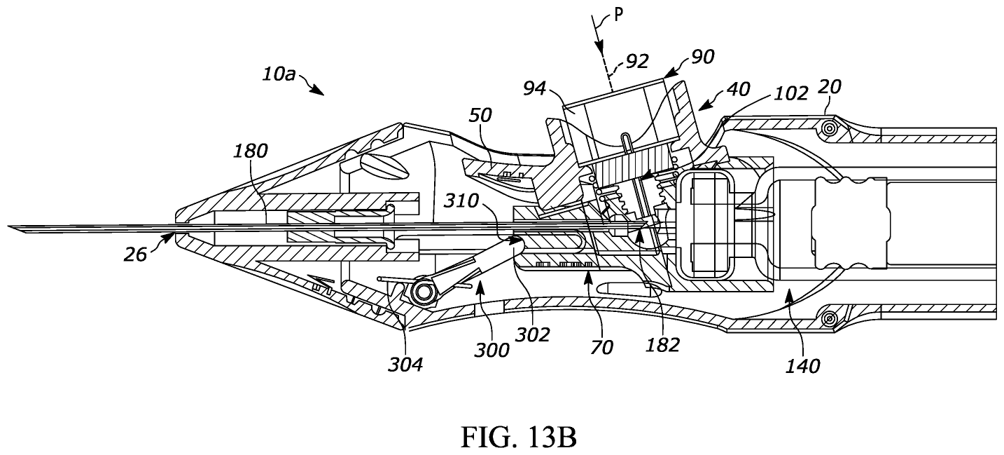

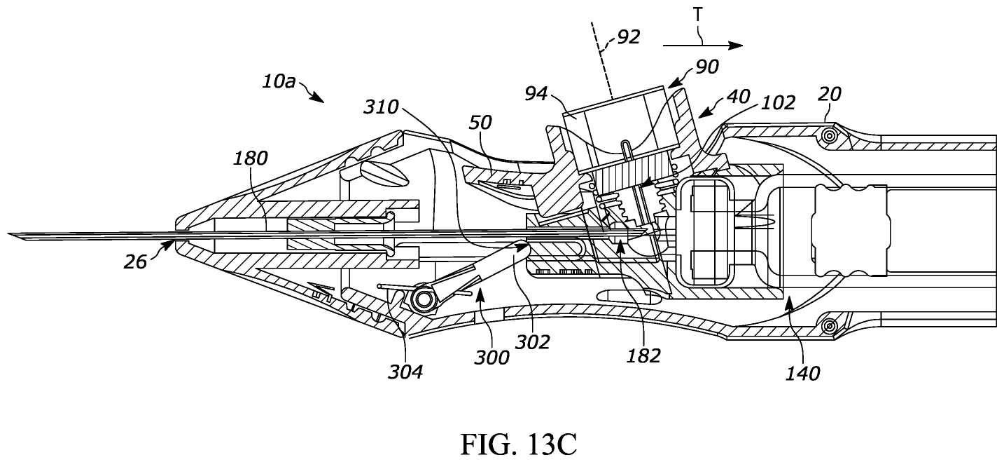

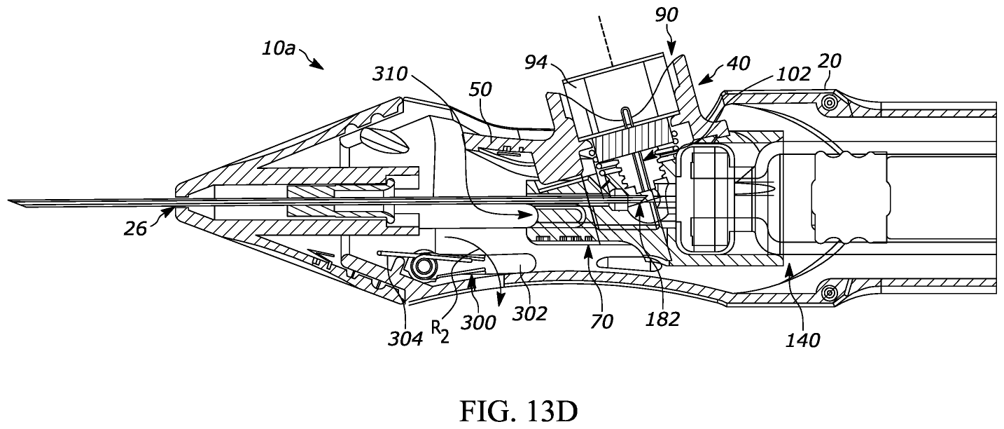

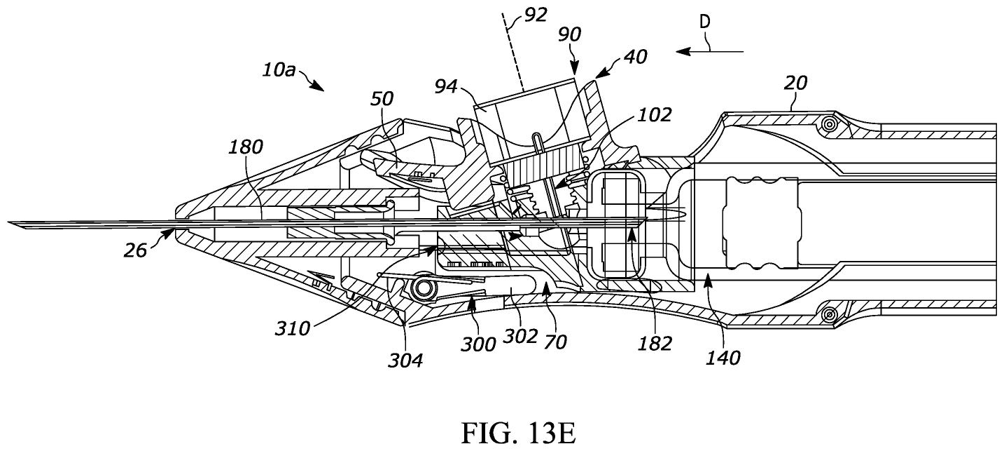

A modified version of the device 10a is illustrated in FIGS. 13A-13E. Referring to FIG. 13A, the device 10a includes a holding mechanism 300 for preventing inadvertent advancing of the advancement clip 70. To this end, the holding mechanism 300 includes a stop bar 302 pivotably connected to the interior of the gripping body 20 by a biasing member 304, such as a spring. The stop bar 302 extends into a recess 310 formed in the distal end of the advancement clip 70. In one example, the recess 310 is positioned below and generally aligned with the center lumen 74 in the advancement clip 70 (see FIG. 5A).

The biasing member 304 biases the stop bar 302 into engagement with the advancement clip 70 (clockwise as shown). Due to this configuration, the advancement clip 70 is prevented from moving distally towards the lumen 26 until/unless the follower 40 is maneuvered in a predetermined manner, as will be described.

Turning to FIG. 13B, the device 10a is initially operated in the same manner as described above, i.e., the follower 40/vacuum sample assembly 90 are collectively depressed in the manner P relative to the advancement clip 70 to automatically draw the vitreous sample into the container 94. At this time, the stop bar 302 is positioned in the recess 310. Both the follower 40 and the advancement clip 70 are then pulled rearwards relative to the gripping body 20 as indicated generally at T in FIG. 13C.

When this occurs, the advancement clip 70 is moved proximally such that the end of the stop bar 302 becomes spaced from the recess 310. Consequently, the spring 304 biases the stop bar 302 to pivot downwards in the manner R₂ and out of the movement path of the advancement clip. In one example, the stop bar 302 pivots in the manner R₂ until it abuts the gripping body 20.

With the stop bar 302 pivoted out of the way, the user can collectively advance the follower 40, advancement clip 70, and vacuum sample assembly 90 in the direction D (FIG. 13E) to position the first end 182 of the needle 180 within the syringe 140 in fluid communication with the drug (not shown).

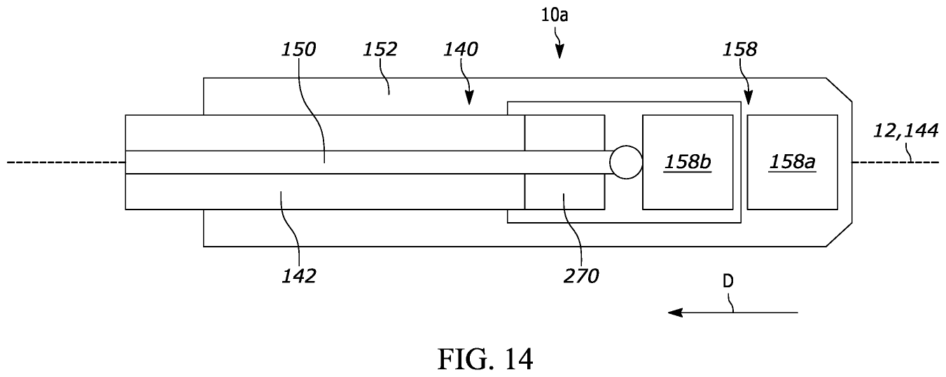

In another example shown in FIG. 14, the biasing feature 158 is separated into two separate portions for isolating different advancement biases/aides from one another. This is schematically illustrated in FIG. 14, in which the portions 158a, 158b are longitudinally aligned with one another but physically separated. In one instance, one portion 158a of the biasing feature 158 helps to advance the syringe 140 and another portion 158b helps to advance the plunger 150 to dispense the drug. T. Alternatively, both portions 158a, 158b can cooperate to advance the syringe 140 and cooperate to advance the plunger 150 relative to the body 142.

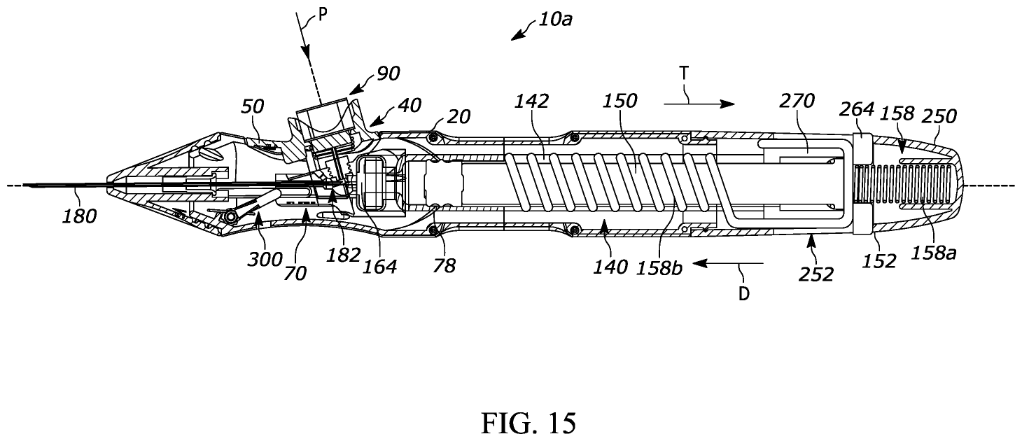

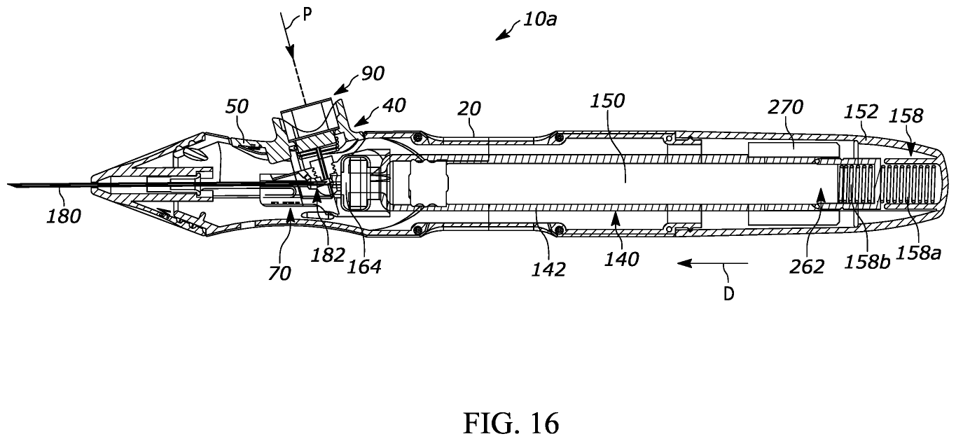

FIGS. 15-16 are specific constructions reflecting the concept shown in FIG. 14. In FIG. 15, the first portion 158a is a compression spring positioned longitudinally between the end cap 250 and the proximal end of the guide body 142. The second portion 158b is a tension spring connected to the receiving member 78 of the advancement clip 70 and the end 262 of the plunger 250.

In operation, the follower 40 is depressed in the manner P to begin vitreous sampling and position the projection 50 within the gripping body 20. The follower 40 is then pulled rearward towards the end cap 250 in the manner T, which compresses the first portion 158a while allowing the holding mechanism 300 to rotate out of engagement with the advancement clip 70.

Thereafter releasing the follower 40 allows the first portion 158a to automatically advance the follower 40, advancement clip 70, vacuum sample assembly 90, and syringe 140 in the direction D. During this initial advancement, the collar 270 rotates as previously described until the projections 264 on the plunger 150 move into and pass through the helical groove(s) 272. This allows the plunger 150 to then move into and relative to the body 142 to dispense the drug V. Simultaneous shortening of the second portion 158b and lengthening of the first portion 158a allows the biasing feature 158 to facilitate expulsion/delivery of the drug. Consequently, a controlled, two-stage biased advancement of the entire syringe 140 and then the plunger 150 occurs.

In FIG. 16, the follower 40 is depressed in the manner P to begin vitreous sampling and position the projection 50 within the gripping body 20. Thereafter releasing the follower 40 allows the first portion 158a to move the syringe 140 by pushing directly against the end of the guide body 142, which has been lengthened and closed at the proximal end. This advances the entire syringe 140 toward the needle 180 until the proximal end 182 thereof pieces the septum 164.

At the same time, the advancement clip 70 bottoms out against the interior of the gripping body 20 to prevent further movement of the syringe 140. Once this occurs, the second portion 158b pushes against the plunger 150 to move the plunger in the manner D relative to the guide body 142 and dispense the drug out through the needle 180. Consequently, a controlled, two-stage biased advancement of the entire syringe 140 and then the plunger 150 occurs. It will be appreciated that the first portion 158a can be omitted (not shown).

In both configurations, each of the portions 158a, 158b can be specifically tuned to provide a desired biasing force as well as a relative biasing force between the portions. That said, the material, number of turns, spring constant, etc. of each portion 158a, 158b can be selected to provide the desired biasing force for each portion 158a, 158b.

Regardless of the configuration of the biasing structure (a single spring, multiple springs, etc.) the biasing structure that provides the force to eject the drug also pushes forward on the syringe 140 and assists the forward motion of the vacuum sample assembly 90, follower 40, and advancement clip 70. With this in mind, it is desirable that the health care provider does not need to apply an excessive amount of force to move the assembly 90 forward through the steps of sample acquisition and drug delivery, so this assistant force is generally desirable.

It is not desirable, however, that the health care provider must provide a strong force to restrain the assembly 90 from moving forward, or that the provider should be surprised by a large amount of force suddenly pulling forward on the assembly. This pulling force should be resisted to allow time for the sample to be acquired. The amount of biasing feature 158 force should also be appropriately matched to a desired delivery time for a drug of a particular viscosity and a needle 180 of a particular length and lumen diameter.

In the case that a stronger biasing feature 158 force is used to achieve a faster drug delivery, the design may be adjusted to balance the forward-directed forces of the spring. To this end, an increased resisting force can be applied by adjusting the properties of the compliant sealing member 130 within the advancement clip 70. For example, increasing the durometer of the compliant sealing member 130 will increase the compression force applied to the procedure needle 180 and therefore increase the friction resisting forward motion.

Alternatively or additionally, adjustments may be made to the advancement clip 70 and/or to the follower 140 interface with the gripping body 20 to increase the friction to resist motion or create a detent to overcome. For example, the bottom surface of the advancement clip 70 may have a small protrusion that interacts with a shelf on the outer housing 20. The size and inclination of the protrusion and its mating surface can be tuned to provide the ideal amount and profile of force to balance the force provided by the biasing feature 158 pushing on the drug cartridge to achieve a comfortable and controllable action of the device 10.

An alternative approach to dealing with high viscosity drug solutions that may be practiced alone or in combination with the above force balancing actions is to adjust the inner diameter of the drug cartridge. Reducing the inner diameter results in a larger pressure on the drug for the same force applied on the plunger 150. In this construction, the plunger 150 will require a longer travel to deliver the same volume of drug. Straightforward adjustments of the rest of the design can be applied to accommodate this change.

In the above descriptions the drug has been packaged within a luer-tipped syringe 140 terminated in a luer-to-vial tip adapter 160, which places a pierceable septum 164 at the tip. This configuration mimics a traditional vial-capped cartridge. In both devices 10 and 10a, the syringe 140 and the adapter 160 can be replaced with a drug cartridge. Straightforward modifications to the geometry of some mating surfaces accommodates this change.

The combined biological sampling and injection assemblies described herein are advantageous in that they provide for a small gauge, e.g., 30G or smaller, pencil-grip instrument that can directly penetrate the sclera, does not require protective measures for the sclera, and can be distally operated in a cordless manner. Furthermore, the assemblies are disarmed until removed from the package and do not pressurize the drug until the plunger advances under the spring bias.

With this in mind, it will be appreciated that the device 10a can also be provided in the package 200 (or a modified version thereof) to prevent depression of the vacuum sample assembly and thereby disarm the device until use/operation is desired.

It will also be appreciated that the device 10, 10a can be configured solely as a vitreous acquisition device. In such configurations, the syringe 140 would be omitted. Moreover, the needle 180 and piercing member 102 could be retained as separate components (as shown) or formed as a single, curved or angled component extending continuously between the ends 104, 184 shown. In other words, the members 102, 180 could be integrated into a single, continuous member.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A vitrectomy device comprising:
a body extending along an axis from a proximal end to a distal end and having a central passage;
a needle for insertion into the vitreous humor of an eye and extending within the central passage and out of the distal end of the body; and
an assembly including a chamber under vacuum and a piercing member extending away from the chamber, wherein the chamber is movable in a direction transverse to the centerline from a first position in which the chamber is not fluidly connected to the needle to a second position in which the piercing member fluidly connects the chamber to the needle for drawing a vitreous sample into the chamber under vacuum pressure.

2. The device recited in claim 1, wherein the chamber is longitudinally movable with the assembly to a third position in which the needle is fluidly connected to a drug to be delivered to the eye.

3. The device recited in claim 2, further comprising a plunger received in the body and longitudinally displaceable relative to the body to force the drug into the eye.

4. The device recited in claim 3, further comprising a biasing feature for biasing the plunger towards the distal end of the body.

5. The device recited in claim 4, further comprising a cam system that cooperates with the biasing feature for preventing pressurization of the drug until after the chamber reaches the third position.

6. The device recited in claim 3, wherein the plunger is part of a syringe including a septum that is pierced by the needle when the chamber moves to the third position to place the needle in fluid communication with the drug.

7. The device recited in claim 6, further comprising a biasing feature having a first portion for biasing the syringe towards the distal end of the body and a second portion for biasing the plunger relative to the syringe towards the distal end of the body.

8. The device recited in claim 2, further comprising a feature for biasing the chamber from the second position to the third position.

9. The device recited in claim 2, wherein the assembly includes a projection for preventing movement of the chamber to the third position until after the chamber is moved to the second position.

10. The device recited in claim 2, further comprising a stop bar pivotably connected to the body and preventing movement of the chamber to the third position until the chamber is first moved longitudinally towards the proximal end of the body.

11. The device recited in claim 2 further comprising:
a sharpened tip provided on the proximal end of the needle;
a compliant member provided in the assembly such that moving the assembly to the third position causes the sharpened tip to pass through the compliant member to stop fluid communication between the piercing member and the proximal end of the needle and establish fluid communication between the needle and the drug.

12. The device recited in claim 1, wherein the distal end of the needle comprises a sharpened tip configured for insertion into or through at least one of the sclera, the pars plana, the anterior segment or the vitreous body of the eye.

13. The device recited in claim 1, wherein the body comprises a pencil-grip type body.

14. The device recited in claim 1, wherein the assembly comprises:
an advancement clip longitudinally slidable within the central passage of the body and including first and second pairs of pockets;
a follower connected to the advancement clip and receiving the vacuum chamber, the follower including first and second projections engaged with the first pockets when the chamber is in the first position and moving to the second position into engagement with the second pockets in response to force applied in the direction transverse to the centerline; and
a piercing member extending through the advancement clip and the follower, the piercing member entering the vacuum chamber when the chamber moves into the second position.

15. The device recited in claim 1, wherein the needle and the piercing member are formed a single, continuous component.

16. The device recited in claim 1, wherein the chamber is removably inserted in the follower for separation of the vitreous sample from the vitrectomy device.

17. A method comprising:
inserting a sharpened distal end of a needle of a device into a globe of an eye, wherein the device comprises:
a body extending along an axis from a proximal end to a distal end and having a central passage;
a needle for insertion into the vitreous humor of an eye and extending within the central passage and out of the distal end of the body; and
an assembly including a chamber under vacuum and a piercing member extending away from the chamber,
moving the chamber in a direction transverse to the centerline from a first position in which the chamber is not fluidly connected to the needle to a second position in which the piercing member fluidly connects the chamber to the needle for drawing a vitreous sample into the chamber under vacuum pressure.

18. The method of claim 16, further comprising releasing the chamber to allow a biasing feature to longitudinally move the chamber with the assembly to a third position in which the needle is fluidly connected to a drug to be delivered to the eye.

19. The method of claim 17, wherein the biasing feature comprises a spring that automatically urges a plunger received in the body and longitudinally displaceable relative to the body to force the drug into the eye once the chamber reaches the third position.

20. The method of claim 16, further comprising pulling the chamber towards proximal end prior to releasing the chamber to allow a stop bar to disengage from the assembly and allow the assembly to move to the third position.
